(19) United States
(12) Patent Application Publication
(10) Pub. No.: US 2003/0162695 A1
(43) Pub. Date: Aug. 28, 2003

(54) Glucocorticoid blocking agents for increasing blood-brain barrier permeability

(76) Inventors: Alan F. Schatzberg, Los Altos, CA (US); Steven Lindley, Redwood City, CA (US); Joseph K. Belanoff, Woodside, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/087,227

(22) Filed: Feb. 27, 2002

(51) Int. Cl. A61K 38/14; A61K 31/7048; A61K 31/704; A61K 31/66; A61K 31/65; A61K 31/56; A61K 31/545; A61K 31/135

(52) U.S. Cl. 514/8; 514/171; 514/27; 514/192; 514/200; 514/105; 514/152; 514/37; 514/29; 514/283; 514/291; 514/651; 424/649; 514/589

(57) ABSTRACT

Glucocorticoid blockers, including glucocorticoid receptor antagonists, are effective to prevent glucocorticoid-induced decrease in permeability of the blood-brain barrier and to increase the permeability of the blood-brain barrier. Administration of glucocorticoid blockers, including glucocorticoid receptor antagonists, concomitant with administration of drugs for treating diseases of the central nervous system increases delivery of such drugs into the central nervous system.

### FIELD OF THE INVENTION

This invention relates to methods and formulations for increasing the permeability of the blood-brain barrier. In particular, this invention relates to methods of using glucocorticoid blockers, such as glucocorticoid receptor antagonists, to increase the permeability of the blood-brain barrier and to pharmaceutical compositions containing glucocorticoid receptor antagonists.

### BACKGROUND OF THE INVENTION

Steroid hormones are well known to have significant effects on animal cells. Corticosteroids are steroid hormones released by the adrenal glands. The most significant human adrenal corticosteroids are cortisol, corticosterone and aldosterone. Based on their observed effects on carbohydrate, mineral and water metabolism, these compounds have been divided into two classes: the mineralocorticoids, affecting mineral and water metabolism, such as aldosterone; and the glucocorticoids, affecting carbohydrate metabolism, such as corticosterone and cortisol (hydrocortisone, 17-hydroxycorticosterone). Corticosterone can act as both a glucocorticoid and as a mineralocorticoid.

Corticosteroids produce cellular effects following binding to receptors located in the cell membrane. Ligand-bound receptors are subsequently internalized and migrate to the nucleus of the cell, where they act on the nuclear material to alter gene expression in the cell. Two general classes of corticosteroid receptors are now recognized, the mineralocorticoid receptors (also termed type I, or MR) and the glucocorticoid receptors (also termed type II, or GR, or cortisol receptors). In addition, it is well known that there are also other steroid receptors which may be present on some animal cells. An example of another steroid hormone receptor is the progesterone receptor.

Mineralocorticoid receptors (MRs) bind corticosterone with high affinity, and bind glucocorticoids with a ten-fold higher affinity than glucocorticoid receptors (GRs) bind glucocorticoids. Thus, the activation of the two classes of receptors may differ depending on the corticosteroid concentration. Blood levels of the glucocorticoid cortisol vary over a wide range during the day. In general, normal cortisol concentrations in the blood range from about 0.5 nM to about 50 nM; however, in response to stress, cortisol concentration may exceed 100 nM.

Glucocorticoid blockers are agents that block or reduce the effects of glucocorticoids. Such interference with glucocorticoid action may, for example, be due to interference with binding of glucocorticoid agonists to glucocorticoid receptors (GR), or to interference with the action of agonist-bound GR at the cell nucleus, or to interference with expression or processing of gene products induced by the action of agonist-bound GR at the nucleus. Glucocorticoid receptor antagonists (GR antagonists) are compounds which inhibit the effect of the native ligand or of glucocorticoid agonists on GR. One mode of action of GR antagonists is to inhibit the binding of GR ligands to GR. A discussion of glucocorticoid antagonists may be found in Agarwal et al. “Glucocorticoid antagonists”, FEBS Lett., 217:221-226 (1987). An example of a GR antagonist is mifepristone, (11, 17)-11-[4-(dimethylmino) phenyl]-17-hydroxy-17-(1-propynyl)estra-4,9-dien-3-one, also known as RU-486 or RU-38486. See U.S. Pat. No. 4,368,085. Mifepristone binds specifically to GR with high affinity (Kd≦10⁻⁹ M). This is an affinity about 18 times that of the affinity of cortisol for GR. GR antagonists may be steroids, such as mifepristone, or non-steroids.

Examples of other steroidal GR antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, and 5,696,127. Such steroidal GR antagonists include cortexolone, dexamethasoneoxetanone, 19-nordeoxycorticosterone, 19-norprogesterone, cortisol-21-mesylate; dexamethasone-21-mesylate, 11β-(4-dimethylaminoethoxyphenyl)-17α-propynyl-17β-hydroxy-4,9-estradien-3-one (RU009), and 17β-hydroxy-17α-19-(4-methylphenyl)androsta-4,9(11)-dien-3-one (RU044).

Examples of other non-steroidal GR antagonists include ketoconazole, clotrimazole; N-(triphenylmethyl)imidazole; N-([2-fluoro-9-phenyl]fluorenyl)imidazole; N([2-pyridyl]diphenylmethyl)imidazole; N-(2-[4,4′,4″-trichlorotrityl]oxyethyl)morpholine; 1-(2[4,4′,4″-trichlorotrityl]oxyethyl)-4-(2-hydroxyethyl)piperazine dimaleate; N-([4,4′,4″]trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1-(2-chlorotrityl)-1,2,4-triazole; 1,S-bis(4,4′,4″-trichlorotrityl)-1,2,4-triazole-3-thiol; and N-((2,6-dichloro-3-methylphenyl)diphenyl)methylimidazole (see U.S. Pat. No. 6,051,573); and the GR antagonist compounds disclosed in U.S. Pat. No. 5,696,127; the compounds disclosed in PCT International Application No. WO 96/19458, which describes non-steroidal compounds which are high-affinity, highly selective antagonists for steroid receptors, such as 6-substituted-1,2-dihydro-N-protected-quinolines; and some opioid ligands, such as the opioid compounds dynorphin-1,13-diamide, U50,488 (trans-(1R,2R)-3,4-dichloro-N-methylN-[2-(1-pyrrolidinyl)cyclohexyl]benzeneacetamide), bremazocine and ethylketocyclazocine; and the non-specific opioid receptor ligand, naloxone, as disclosed in Evans et al., Endocrin., 141:2294-2300 (2000).

The disclosures of all patents, patent applications, and other documents cited in this application are incorporated by reference.

It has long been recognized that the central nervous system (CNS) is a privileged compartment within an animal, and that transport between the blood and the CNS is less rapid, more difficult and more closely regulated than transport between the blood and other body compartments. The “blood-brain barrier” (“BBB”) is the term used to describe this functional barrier between the central nervous system and the blood of an animal.

The BBB is affected by corticosteroids. For example, corticosteroids are reported to decrease BBB permeability (Hedley-Whyte et al., Ann. Neurol. 19:373-377 (1986); Neuwelt et al., J. Neurosurg. 72:123-126 (1990); Paul et al., Int. J. Immunopharm. 17:497-503 (1995); Ziylan et al., J. Neurochem. 51:1338-1342 (1988), Ziylan et al., J. Neurochem. 52:684-689 (1989) and Ziylan et al., Mol. and Chem. Neuropath. 20:203-218 (1993)). Adrenalectomy, which lowers corticosteroid levels, increases BBB permeability (Brown et al., Tox. and Appl. Pharm. 150:158-165 (1988) and Long et al., Science 227:1580-1583 (1985)).

It is well established that stress, whether physical stress such as disease, injury or exercise, or psychological stress, such as anxiety, depression, or fear, leads to increased corticosteroid levels.

Although it is believed that the BBB serves a protective function under normal conditions by protecting the CNS from exposure to potentially toxic compounds, in CNS disease the BBB may thwart therapeutic efforts by hindering the entry of therapeutic compounds into the CNS. For example, although many bacterial and fungal infections may be readily treated where the site of the infection is outside the CNS, such infections in the CNS are often very dangerous and very difficult to treat due to the inability to deliver effective doses of drugs to the site of the infection. Similarly, the action of the BBB makes treatment of cancer of the brain more difficult than treatment of cancers located outside the CNS. Even where it may be possible to deliver an effective dose of drug into the CNS by administering very large amounts of drug outside of the CNS, the drug levels outside the CNS (such as in the blood) are then often so high as to reach toxic levels deleterious to the kidneys, liver, and other vital organs. Accordingly, there is need in the art for methods to improve the delivery of compounds into the CNS.

### SUMMARY OF THE INVENTION

The current invention provides methods of increasing the permeability of the blood-brain barrier of a patient having a CNS disorder.

As such, in one embodiment, the invention provides a method of increasing the permeability of the blood brain barrier in a patient having a CNS disorder amenable to drug therapy and not otherwise indicative of an antiglucocorticoid therapy. This method includes administering an antiglucocorticoid drug and a therapeutic drug to the patient. The amount of antiglucocorticoid administered in this method is sufficient to increase the permeability of the blood brain barrier to the therapeutic drug.

In another embodiment, this invention provides a method of increasing the amount of a therapeutic drug delivered to the CNS of a patient having a CNS disorder amenable to drug therapy and not otherwise indicative of an antiglucocorticoid therapy. This method includes administering an antiglucocorticoid drug and a therapeutic drug to the patient. The amount of antiglucocorticoid administered in this method is sufficient to increase the permeability of the blood brain barrier to the therapeutic drug.

In some embodiments, the CNS disorder is a neoplastic disease, a bacterial disease, a viral disease, a fungal disease, a neuropsychiatric disease or a neurodegenerative disorder.

In one embodiment, the CNS disorder is a neoplastic disease and the therapeutic drug is a chemotherapeutic agent. In one aspect, the chemotherapeutic agent is administered in combination with radiation therapy. In some embodiments, the neoplastic disease is a cerebral metastases or malignant astrocytoma. In some embodiments, the chemotherapeutic agent is a nitrosoureas. In one aspect, the nitrosoureas is lomustine. In another aspect, the nitrosoureas is semustine. In yet another aspect, the nitrosoureas is carmustine.

In one embodiment, the CNS disorder is a cerebral metastases and the chemotherapeutic agent is a microtubilin inhibitor. In another aspect, the chemotherapeutic agent is a topoisomerase inhibitor, an antimicrobial agent or a platinum compound. In another aspect the chemotherapeutic agent is vinblastine, etoposide, topotecan, penicillin, or cisplatin.

In one embodiment of the present invention, the CNS disorder is a cerebral metastases and the chemotherapeutic agent is an antimetabolite. In another embodiment, the chemotherapeutic agent is a DNA damaging agent, endocrine agent or antitumor antibiotic. In another embodiment, the chemotherapeutic agent is methotrexate, cyclophosphamide, bleomycin or tamoxifen.

In some embodiments of the invention, the CNS disorder is a bacterial disease In one aspect, the bacterial disease is bacterial meningitis. In another aspect, the bacterial disease is a bacterial abscess. In one embodiment, the therapeutic drug is selected from the group consisting of penicillins, cephalosporins, monobactams, carbapenems, aminoglycosides, glycopeptides, tetracyclines, macrolides, sulfonamides, trimethoprims, and chloramphenicol.

In one embodiment of the present invention, the bacterial disease is bacterial meningitis and the therapeutic drug is chloramphenicol. In another embodiment, the drug is ampicillin, cefotaxime, ceftriaxone or fectizoxime.

In another embodiment, the bacterial disease is a CNS bacterial abscess and the therapeutic drug is chloramphenicol. In another embodiment, the drug is penicillin or metronidazole.

In some embodiments of the present invention, the CNS disorder is viral encephalitis. In one aspect, the CNS disorder is HSV encephalitis. In another aspect, the CNS disorder is cytomegalovirus encephalitis. In yet another aspect, the CNS disorder is varicella encephalitis. In one embodiment, the CNS disorder is viral and the therapeutic drug is acyclovir. In another embodiment, the therapeutic drug is ganciclovir or foscarnet.

In one embodiment of the present invention, the CNS disorder is HIV encephalitis and the therapeutic drug is acyclovir. In another embodiment, the therapeutic drug is ceftriaxone, pyrimethamine, sulfadiazine, clindamycin, flucytosine, doxycycline, ganciclovir or foscarnet.

In some embodiments, the CNS disorder is a neuropsychiatric disease. In one aspect, the neuropsychiatric disease is a psychotic disorder. In another aspect, the neuropsychiatric disease is an affective disorder. In one embodiment, the neuropsychiatric disease is major depression, mania, or bipolar manic-depressive illness and the therapeutic drug is an antidepressant, anticonvulsant, or antipsychotic agent. In another embodiment, the neuropsychiatric disease is schizophrenia, schizoaffective disorder or panic/anxiety disorder and the therapeutic drug is an antidepressant, anticonvulsant, or antipsychotic agent. In one embodiment, the antidepressant is a fluoxetine-selective serotonin reuptake inhibitor, an amitriptyline-tricyclic or venlafaxine-bupropion. In another embodiment, the antipsychotic is Haloperidol, Risperidone, or Olanzapine. In yet another embodiment, the anticonvulsant is Valproic acid, Topiramate, Carbamazepine, or Lithium. In even another embodiment, the therapeutic drug is Lorazepam, Clonazepam, or Buspirone.

In some embodiments, the CNS disorder is a neurodegenerative disorder. In one aspect, the neurodegenerative disorder is Alzheimer's Disease. In another, the neurodegenerative disorder is multiple sclerosis, Parkinson's Disease, or seizure disorder. In one embodiment, the neurodegenerative disorder is Alzheimer's Disease and the therapeutic drug is an acetylcholinesterase inhibitor. In another embodiment, the neurodegenerative disorder is multiple sclerosis and the therapeutic drug is Interferon-1b, Interferon-1a, or glatiramer acetate. In yet another embodiment, the neurodegenerative disorder is seizure disorder and the therapeutic drug is carbamazepine, fosphenytoin, valproic acid, phenytoin, felbamate, clonazepam, primidone, topiramate, ehthosuximide, gabapentin or phenobarbitol. In even another embodiment, the neurodegenerative disorder is Parkinson's Disease and the therapeutic drug is levodopa, carbidopa, benserazide, pergolide, bromocriptine, selegiline, amantadine, or trihexyphenidyl HCL.

In some embodiments, the CNS disorder is fungal. In one aspect, the CNS disorder is fungal and the therapeutic drug is amphotericin B, flucytosine, fluconazole, or itraconazole.

The current invention also provides kits for the treatment of a patient having a CNS disorder amenable to drug therapy and not otherwise indicative of an antiglucocorticoid therapy. The kit of the present invention includes an antiglucocorticoid in sufficient amount to increase permeability of the patient's blood brain barrier, a therapeutically effective amount of a drug useful for treating the CNS disorder, and instructions for the concomitant administration of the drug and the antiglucocorticoid. In some embodiments of the present invention, the CNS disorder is a neoplastic disease, bacterial disease, viral disease, fungal disease, neuropsychiatric disease, or neurodegenerative disorder.

### DETAILED DESCRIPTION OF THE INVENTION

I. Introduction

This invention relates to methods and formulations for increasing the permeability of the blood-brain barrier. Inhibition of the action of glucocorticoid receptors is effective to increase the permeability of the BBB. Increased BBB permeability is effective to increase the delivery of therapeutic drugs into the CNS.

In the present invention, an antiglucocorticoid is administered concomitantly with a therapeutic drug to a patient having a CNS disorder amenable to drug therapy and not otherwise indicative of an antiglucocorticoid therapy. Administration of an antiglucocorticoid concomitantly with a therapeutic drug to a patient having a CNS disorder increases the permeability of the blood-brain barrier of the patient thereby permitting the therapeutic drug to reach higher concentrations in the CNS.

Using the methods of the present invention, therapeutic drugs previously not used for treating patients having CNS disorders because of the drug's inability to reach sufficient concentration in the CNS of a patient may be used in combination with an antiglucocorticoid therapy. Additionally, therapeutic drugs currently used for treating patients having CNS disorders may be administered at lower dosages when administered in combination with an antiglucocorticoid. At lower dosages, the therapeutic drug may be less toxic to the patient thereby providing more effective treatment with less risk to the patient.

II. Definitions

“Animal” includes humans and non-human mammals, such as companion animals (cats, dogs, and the like) and farm animals (cattle, horses, sheep, goats, swine, and the like).

“Disease” includes any unhealthy condition of an animal, including particularly tumors, especially tumors of the internal organs, and parasitic, bacterial, fungal, and viral infections.

“CNS disease” means any unhealthy condition of the central nervous system (CNS) of an animal. An unhealthy condition may be the result of the presence of undesirable organisms, such as bacteria, fungi, viruses, or other disease-causing organisms, or may be the result of the presence of undesirable cells, such as malignant cells, or excessive white blood cells, or other cells whose presence causes a disease condition, or may be the result of the presence of undesirable materials, such as toxins, metals, metabolites, peptides, plaques, or other materials, or may be a neurodegenerative condition or a condition of unknown origin, such as psychosis, schizophrenia, depression, or other psychiatric condition.

The term “glucocorticoid receptor” (abbreviated “GR”) denotes a molecule or molecules that bind glucocorticoids with high affinity; in particular, GR refers to the type II corticosteroid receptor.

The term “glucocorticoid blocker” denotes a molecule or molecules that block or reduce the effects of glucocorticoids. Any compound effective to antagonize glucocorticoid action is a glucocorticoid blocker.

The term “glucocorticoid receptor antagonist” (abbreviated “GR antagonist”) denotes compounds which inhibit the effect of the native ligand or of GR agonists on GR. GR antagonists are glucocorticoid blockers.

“Concomitant administration” of a drug with a glucocorticoid blocker means administration of the drug and the glucocorticoid blocker at such times that the drug is present in the blood at such a level that the drug can reach a therapeutically effective level in the CNS when the BBB is lowered by a BBB-lowering amount of a glucocorticoid blocker. Such concomitant administration may involve concurrent (i.e. at the same time), prior or subsequent administration of the drug with respect to the administration of a glucocorticoid blocker, depending on the onsets of action and half-lives of the drug and glucocorticoid blocker chosen. A person of ordinary skill in the art, having knowledge of the drugs and glucocorticoid blockers, would have no difficulty determining the appropriate timing, sequence and dosages of administration for particular drugs and glucocorticoid blockers.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Such salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester may be a mono-acid-mono-salt or ester or a di-salt or ester; and similarly where there are more than two acidic groups present, some or all of such groups can be salified or esterified. Compounds named in this invention may be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

A “therapeutically effective amount” means the amount that, when administered to an animal for treating a disease, is sufficient to effect treatment for that disease. In the case of a drug for treating a disease of the CNS that is concomitantly administered with a BBB-permeability-increasing effective amount of a glucocorticoid blocker, the therapeutically effective amount of the drug when concomitantly administered with the glucocorticoid blocker will be lower than the therapeutically effective amount of the drug when not concomitantly administered with the glucocorticoid blocker. Thus, a drug concomitantly administered with a glucocorticoid blocker may be effective in treating a CNS disease at a blood level that would be ineffective in the absence of the effects of the glucocorticoid blocker.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

III. Steroidal Anti-Glucocorticoids as Glucocorticoid Blockers

In one embodiment of the invention, steroidal glucocorticoid blockers are administered to increase BBB permeability. Steroidal antiglucocorticoids, such as steroidal GR antagonists, can be obtained by modification of the basic structure of glucocorticoid agonists, i.e., varied forms of the steroid backbone. The structure of cortisol can be modified in a variety of ways. The two most commonly known classes of structural modifications of the cortisol steroid backbone to create glucocorticoid blockers include modifications of the 11β-hydroxy group and modification of the 17β side chain. (Lefebvre et al., J. Steroid Biochem. 33:557-563 (1989)).

IV. Removal or Substitution of the 11-β Hydroxy Group

In another embodiment of the invention, glucocorticoid agonists with modified steroidal backbones comprising removal or substitution of the 11β-hydroxy group are administered. This class includes natural antiglucocorticoids, including cortexolone, progesterone and testosterone derivatives, and synthetic compositions, such as mifepristone (Lefebvre, et al., cited above). Preferred embodiments of the invention include all 11β-aryl steroid backbone derivatives because these compounds are devoid of progesterone receptor (PR) binding activity (Agarwal, cited above). Another preferred embodiment comprises an 11β-[4-(dimethylamino)phenyl] steroid backbone derivative, i.e., mifepristone, which is both an effective anti-glucocorticoid and anti-progesterone agent. These compositions act as reversibly-binding steroid receptor antagonists. For example, when bound to a 11β-[4(dimethylamino)phenyl] steroid, the steroid receptor is maintained in a conformation that cannot bind its natural ligand, such as cortisol in the case of GR (Cadepond, et al., Ann. Rev. Med. 48:129 (1997)).

Synthetic 11-β phenyl-aminodimethyl steroids include mifepristone, also known as RU486, or 17-β-hydroxy-11-β-(4-dimethyl-aminophenyl)17-α-(1-propynyl)estra-4,9-dien-3-one). It has been shown to be both a powerful progesterone receptor antagonist and a powerful GR antagonist. Other 11-β phenyl-aminodimethyl steroids shown to have GR antagonist effects include RU009 (RU39.009), 11-β-(4-dimethyl-aminoethoxyphenyl)-17-α-(propynyl-17 β-hydroxy-4,9-estradien-3-one) (see Bocquel (1993) J. Steroid Biochem. Molec. Biol. 45:205-215). Another GR antagonist related to RU486 is RU044 (RU43.044) 17-β-hydroxy-17-α-19-(4-methyl-phenyl)-androsta-4,9(11)-dien-3-one) (Bocquel (1993)supra). See also Teutsch (1981) Steroids 38:651-665: U.S. Pat. Nos. 4,386,085 and 4,912,097.

Some GR antagonist compounds containing the basic glucocorticoid steroid structure are irreversible anti-glucocorticoids. Such compounds include α-keto-methanesulfonate derivatives of cortisol, including cortisol-21-mesylate (4-pregnene-11-β, 17-α,21-triol-3,20-dione-21-methane-sulfonate and dexamethasone-21-mesylate (16-methyl-9-α-fluoro-1,4-pregnadiene-11 β, 17-α, 21-triol-3,20-dione-21-methane-sulfonate). See Simons (1986) J. Steroid Biochem. 24:25-32 (1986); Mercier (1986) J. Steroid Biochem. 25:11-20; U.S. Pat. No. 4,296,206.

V. Modification of the 17-β Side Chain Group

Steroidal antiglucocorticoids which can be obtained by various structural modifications of the 17-β side chain are also used in the methods of the invention. This class includes synthetic antiglucocorticoids such as dexamethasone-oxetanone, various 17, 21-acetonide derivatives and 17-β-carboxamide derivatives of dexamethasone (Lefebvre, et al. (1989) supra; Rousseau (1979) Nature 279:158-160).

VI. Other Steroid Backbone Modifications

GR antagonists used in the various embodiments of the invention include any steroid backbone modification which effects a biological response resulting from a GR-agonist interaction. Steroid backbone antagonists can be any natural or synthetic variation of cortisol, such as adrenal steroids missing the C-19 methyl group, such as 19-nordeoxycorticosterone and 19-norprogesterone (Wynne (1980) Endocrinology 107:1278-1280).

In general, the 11-β side chain substituent, and particularly the size of that substituent, can play a key role in determining the extent of a steroid's anti glucocorticoid activity. Substitutions in the A ring of the steroid backbone can also be important. 17-hydroxypropenyl side chains generally decrease antiglucocorticoidal activity in comparison to 17-propynyl side chain containing compounds.

VII. Non-Steroidal Anti-Glucocorticoids as Glucocorticoid Blockers

Non-steroidal glucocorticoid antagonists are also used in the methods of the invention to increase BBB permeability or to prevent a decrease in BBB due to glucocorticoid action. These include synthetic mimetics and analogs of proteins, including partially peptidic, pseudopeptidic and non-peptidic molecular entities. For example, oligomeric peptidomimetics useful in the invention include (α-β-unsaturated) peptidosulfonamides, N-substituted glycine derivatives, oligo carbamates, oligo urea peptidomimetics, hydrazinopeptides, oligosulfones and the like (de Bont (1996) Bioorganic & Medicinal Chem. 4:667-672). The creation and simultaneous screening of large libraries of synthetic molecules can be carried out using well-known techniques in combinatorial chemistry, for example, see van Breemen (1997) Anal Chem 69:2159-2164; Lam (1997) Anticancer Drug Des 12:145-167 (1997). Peptidomimetics specific for GR can be designed using computer programs in conjunction with combinatorial chemistry, (combinatorial library) screening approaches (Murray (1995) J. Computer-Aided Molec. Design 9:381-395); Bohm (1996) J. Computer-Aided Molec. Design 10:265-272). Such “rational drug design” can help develop peptide isomerics and conformers including cycloisomers, retro-inverso isomers, retro isomers and the like (as discussed in Chorev (1995) TibTech 13:438-445).

VIII. Identifying Glucocorticoid Blockers

Because any glucocorticoid blocker can be used for increasing the permeability of the BBB or preventing or reducing glucocorticoid-induced decreases in BBB permeability in the methods of the invention, in addition to the compounds and compositions described above additional useful glucocorticoid blockers can be determined by the skilled artisan. A variety of such routine, well-known methods can be used and are described in the scientific and patent literature. They include in vitro and in vivo assays for the identification of additional glucocorticoid blockers. A few illustrative examples are described below.

One assay that can be used to identify a GR antagonist of the invention measures the effect of a putative GR antagonist on tyrosine amino-transferase activity in accordance with the method of Granner, Meth. Enzymol. 15:633, 1970. This analysis is based on measurement of the activity of the liver enzyme tyrosine amino-transferase (TAT) in cultures of rat hepatoma cells (RHC). TAT catalyzes the first step in the metabolism of tyrosine and is induced by glucocorticoids (cortisol) both in liver and hepatoma cells. This activity is easily measured in cell extracts. TAT converts the amino group of tyrosine to 2-oxoglutaric acid. P-hydroxyphenylpyruvate is also formed. It can be converted to the more stable p-hydroxybenzaldehyde in an alkaline solution and quantitated by absorbance at 331 nm. The putative GR antagonist is co-administered with cortisol to whole liver, in vivo or ex vivo, or hepatoma cells or cell extracts. A compound is identified as a GR antagonist when its administration decreases the amount of induced TAT activity, as compared to control (i.e., only cortisol or GR agonist added) (see also Shirwany, Biochem. Biophys. Acta 886:162-168, 1986).

Further illustrative of the many assays which can be used to identify compositions utilized in the methods of the invention, in addition to the TAT assay, are assays based on glucocorticoid activities in vivo. For example, assays that assess the ability of a putative GR antagonist to inhibit uptake of ³H-thymidine into DNA in cells which are stimulated by glucocorticoids can be used. Alternatively, the putative GR antagonist can complete with ³H-dexamethasone for binding to a hepatoma tissue culture GR (see, e.g., Choi, et al., Steroids 57:313-318, 1992). As another example, the ability of a putative GR antagonist to block nuclear binding of ³H-dexamethasone-GR complex can be used (Alexandrova et al., J. Steroid Biochem. Mol. Biol. 41:723-725, 1992). To further identify putative GR antagonists, kinetic assays able to discriminate between glucocorticoid agonists and antagonists by means of receptor-binding kinetics can also be used (as described in Jones, Biochem J. 204:721-729, 1982).

In another illustrative example, the assay described by Daune, Molec. Pharm. 13:948-955, 1977; and in U.S. Pat. No. 4,386,085, can be used to identify antiglucocorticoid activity. Briefly, the thymocytes of surrenalectomized rats are incubated in nutritive medium containing dexamethasone with the test compound (the putative GR antagonist) at varying concentrations. ³H-uridine is added to the cell culture, which is further incubated, and the extent of incorporation of radiolabel into polynucleotide is measured. Glucocorticoid agonists decrease the amount of ³H-uridine incorporated. Thus, a GR antagonist will oppose this effect.

For additional compounds that can be utilized in the methods of the invention and methods of identifying and making such compounds, see U.S. Pat. Nos. 4,296,206 (see above); 4,386,085 (see above); 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; and 5,616,458; and WO 96/19458, which describes non-steroidal compounds which are high-affinity, highly selective modulators (antagonists) for steroid receptors, such as 6-substituted-1,2-dihydro N−1 protected quinolines.

The specificity of the antagonist for the GR relative to the MR can be measured using a variety of assays known to those of skill in the art. For example, specific antagonists can be identified by measuring the ability of the antagonist to bind to the GR compared to the MR (see, e.g., U.S. Pat. Nos. 5,606,021; 5,696,127; 5,215,916; 5,071,773). Such an analysis can be performed using either direct binding assay or by assessing competitive binding to the purified GR or MR in the presence of a known antagonist. In an exemplary assay, cells that are stably expressing the glucocorticod receptor or mineralocorticoid receptor (see, e.g., U.S. Pat. No. 5,606,021) at high levels are used as a source of purified receptor. The affinity of the antagonist for the receptor is then directly measured. Those antagonists that exhibit at least a 100-fold higher affinity, often 1000-fold, for the GR relative to the MR are then selected for use in the methods of the invention.

A GR-specific antagonist may also be defined as a compound that has the ability to inhibit GR-mediated activities, but not MR-mediated activities. One method of identifying such a GR-specific antagonist is to assess the ability of an antagonist to prevent activation of reporter constructs using transfection assays (see, e.g., Bocquel et al, J. Steroid Biochem Molec. Biol. 45:205-215, 1993, U.S. Pat. Nos. 5,606,021, 5,929,058). In an exemplary transfection assay, an expression plasmid encoding the receptor and a reporter plasmid containing a reporter gene linked to receptor-specific regulatory elements are cotransfected into suitable receptor-negative host cells. The transfected host cells are then cultured in the presence and absence of a hormone, such as cortisol or analog thereof, able to activate the hormone responsive promoter/enhancer element of the reporter plasmid. Next the transfected and cultured host cells are monitored for induction (i.e., the presence) of the product of the reporter gene sequence. Finally, the expression and/or steroid binding-capacity of the hormone receptor protein (coded for by the receptor DNA sequence on the expression plasmid and produced in the transfected and cultured host cells), is measured by determining the activity of the reporter gene in the presence and absence of an antagonist. The antagonist activity of a compound may be determined in comparison to known antagonists of the GR and MR receptors (see, e.g., U.S. Pat. No. 5,696,127). Efficacy is then reported as the percent maximal response observed for each compound relative to a reference antagonist compound. A GR-specific antagonist is considered to exhibit at least a 100-fold, often 1000-fold or greater, activity towards the GR relative to the MR.

IX. Presently Preferred Compounds

While the broadest definition of the invention is set out in the Summary of the Invention, and it is herein taught that any glucocorticoid blocker, such as, for example, a compound effective to antagonize glucocorticoid binding at a GR, is suitable for the practice of the invention, including all glucocorticoid blockers named herein, both supra and infra, certain GR antagonist compounds as taught in this invention are presently preferred.

Suitable GR antagonist compounds include mifepristone, cortexolone, dexamethasone-oxetanone, 19-nordeoxycorticosterone, 19-norprogesterone, cortisol-21-mesylate; dexamethasone-21-mesylate, 11β-(4-dimethylaminoethoxyphenyl)-17α-propynyl-17β-hydroxy-4,9-estradien-3-one (RU009), and 17β-hydroxy-17α-19-(4-methylphenyl)androsta-4,9(11)-dien-3-one (RU044).

Suitable other steroidal GR antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, and 5,696,127.

Suitable non-steroidal GR antagonists include ketoconazole, clotrimazole; N-(triphenylmethyl)imidazole; N-([2-fluoro-9-phenyl]fluorenyl)imidazole; N-([2pyridyl]diphenylmethyl)imidazole; N-(2-[4,4′,4″-trichlorotrityl]oxyethyl)morpholine; 1-(2[4,4′,4″-trichlorotrityl]oxyethyl)-4-(2-hydroxyethyl)piperazine dimaleate; N-([4,4′,4″]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1-(2-chlorotrityl)-1,2,4-triazole; 1,S-bis(4,4′,4″-trichlorotrityl)-1,2,4-triazole-3-thiol; and N-((2,6-dichloro-3-methylphenyl)diphenyl)methylimidazole (see U.S. Pat. No. 6,051,573); and the GR antagonist compounds disclosed in U.S. Pat. No. 5,696,127; the compounds disclosed in PCT International Application No. WO 96/19458, which describes non-steroidal compounds which are high-affinity, highly selective antagonists for steroid receptors, such as 6-substituted-1,2-dihydro-N-protected-quinolines; and some opioid ligands, such as the opioid compounds dynorphin-1,13-diamide, U50,488 (trans-(1R,2R)-3,4-dichloro-N-methyl-N-[2-(1-pyrrolidinyl)cyclohexyl]benzeneacetamide), bremazocine and ethylketocyclazocine; and the non-specific opioid receptor ligand, naloxone, as disclosed in Evans et al., Endocrin., 141:2294-2300 (2000).

Presently, the preferred glucocorticoid blocker is the GR antagonist mifepristone.

X. Pharmaceutical Compositions and Administration

In general, glucocorticoid blockers suitable for use in the practice of this invention will be administered in therapeutically effective amounts by any of the usual modes known in the art, either singly or in combination with at least one other compound of this invention and with at least one other conventional therapeutic agent for the disease being treated. A therapeutically effective amount may vary widely depending on the disease, its severity, the age and relative health of the animal being treated, the potency of the compound(s), and other factors. Therapeutically effective amounts of glucocorticoid blockers suitable for practice of the method of the invention may range from about 0.5 to about 25 milligrams per kilogram (mg/kg). A person of ordinary skill in the art will be able without undue experimentation, having regard to that skill and this disclosure, to determine a therapeutically effective amount of a particular glucocorticoid blocker compound for practice of this invention.

In general, glucocorticoid blocker compounds may be administered as pharmaceutical compositions by any method known in the art for administering therapeutic drugs to treat disorders of the CNS including oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Alfonso AR: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols.

Aqueous suspensions of the invention contain a GR antagonist in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a GR antagonist in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

Glucocorticoid blocker pharmaceutical formulations can be prepared according to any method known to the art for the manufacture of pharmaceuticals. Such drugs can contain sweetening agents, flavoring agents, coloring agents and preserving agents. Any glucocorticoid blocker formulation can be admixtured with nontoxic pharmaceutically acceptable excipients which are suitable for manufacture.

Typically, glucocorticoid blocker compounds suitable for use in the practice of this invention will be administered orally. The amount of a compound of this invention in the composition may vary widely depending on the type of composition, size of a unit dosage, kind of excipients, and other factors well known to those of ordinary skill in the art. In general, the final composition may comprise from 0.000001 percent by weight (%w) to 10 % w of the glucocorticoid blocker compounds, preferably 0.00001% w to 1% w, with the remainder being the excipient or excipients. For example, the GR antagonist mifepristone is given orally in tablet form, with dosages in the range of between about 0.5 and 25 mg/kg, more preferably between about 0.75 mg/kg and 15 mg/kg, most preferably about 10 mg/kg.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Pharmaceutical preparations for oral use can be obtained through combination of glucocorticoid blocker compounds with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxypropylmethylcellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

The GR antagonists of this invention can also be administered in the form of suppositories for rectal administration of the drug. These formulations can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The GR antagonists of this invention can also be administered by in intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The GR antagonists of the invention can be delivered by transdermally, by a topical route, formulated as applicator sticks, solutions, suspensions, emulsions, gels, creams, ointments, pastes, jellies, paints, powders, and aerosols.

The GR antagonists of the invention can also be delivered as microspheres for slow release in the body. For example, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The GR antagonist pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use

In another embodiment, the GR antagonist formulations of the invention are useful for parenteral administration, such as intravenous (IV) administration. The formulations for administration will commonly comprise a solution of the GR antagonist (e.g., mifepristone) dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of GR antagonist in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the GR antagonist formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR antagonist into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

A pharmaceutical composition of the invention may optionally contain, in addition to a glucocorticoid blocker compound, at least one other therapeutic agent useful in the treatment of a disease or condition of the CNS. Such other compounds may be of any class of drug or pharmaceutical agent, including but not limited to antibiotics, anti-parasitic agents, antifungal agents, anti-viral agents, anti-tumor agents, anti-neurodegenerative agents and anti-psychotic agents. When administered with anti-parasitic, anti-bacterial, anti-fungal, anti-tumor, anti-viral agents, anti-neurodegenerative, and anti-psychotic agents and the like, glucocorticoid blocker compounds may be administered by any method and route of administration suitable to the treatment of the disease, typically as pharmaceutical compositions.

Methods of treating various CNS disorders are well known in the art, See Goodman & Gilmans's, The Pharmalogical Basis of Therapeutics, 9^{th} Edition. Using the methods of the present invention, antiglucocorticoids can be administered in combination with other known therapies to treat patient having CNS disorders. In some instances, drugs previously thought of as not therapeutically effective for treatment of a CNS disorder will be therapeutically effective in combination with an antiglucocorticoid therapy. In other instances, drugs currently used to treat a CNS disorder will be effective at lower dosages than would be possible in the absence of a glucocorticoid blocker. For example, the antibacterial agent gentamicin is currently used to treat a variety of CNS and non-CNS bacterial diseases. Gentamicin's antibacterial actions are dose dependent and at high doses, gentamicin is associated with toxic side effects including nephrotoxicity and ototoxicity. Gentamicin concentrations achieved in the CNS are less than 10% of those achieved in plasma. Gentamicin, however, in combination therapy with an antiglucocorticoid, can achieve higher CNS concentrations at lower dosages thereby reducing negative side effects while increasing efficacy. In another example, the antiviral agent, acyclovir, is currently used to treat viral CNS disorders such as viral encephalitis. The administration of acyclovir to patients suffering from non-CNS viral disorders is about 15 mg/kg per day whereas for patients suffering from viral encephalitis, the necessary therapeutic dosage doubles. Acyclovir concentrations reached in the CNS are 30-50% of those achieved in plasma. In combination therapy with an antiglucocorticoid, the therapeutic dosage of acyclovir for a patient suffering from viral encephalitis may be reduced. In one embodiment of the present invention, the dosage may be reduced from 10-50%.

The methods of the present invention, e.g., treating patients having CNS disorders by administering an antiglucocorticoid to decrease the permeability of the blood brain barrier and concomitantly administering a therapeutic drug for delivery to the patient's CNS, can be used to treat patients having any CNS disorder amenable to drug therapy. In some embodiments of the present invention, CNS disorders amenable to treatment by the methods of the present invention include, for example, neoplastic diseases, bacterial diseases, fungal diseases, neuropsychiatric diseases and neurodegenerative diseases.

In one embodiment of the present invention, the neoplastic diseases treatable include, for example, cerebral metastases and malignant astrocytomas. Methods of treating patients having neoplastic diseases are known in the art. One of skill, for example, will know how to choose an appropriate treatment for use with an antiglucocorticoid of the present invention based on different factors including cell type, stage of the disease, and tumor location and density. In one embodiment, therapeutic drugs useful for treating neoplastic diseases include chemotherapeutic agents, alone, or in combination with, radiation treatment.

Chemotherapeutic drugs useful in treating neoplastic diseases include alkylating agents, antimetabolites, natural products, hormones and antagonists and miscellaneous agents. For example, nitrosoureas are useful in the treatment of brain tumors. In particular, carmustine, semustine, and lomustine are used for the treatment of brain cancers because of their capacity to cross the blood brain barrier. Concomitantly administered with an antiglucocorticoid, these agents will have improved capacity to enter the CNS of a patient. Other chemotherapeutic agents, not known for their ability to cross the blood brain barrier, may be used in the present invention. In some embodiments, the chemotherapeutic agents useful in the present invention will include microtubilin inhibitors, topoisomerase inhibitors, antimicrobial agents, platinum compounds, antimetabolites, DNA damaging agents, endocrine agents or anti-tumor antibiotics. Examples of therapeutic drugs that can be used include vinblastine, etoposide, topotecan, penicillin, cisplatin, methotrexate, cyclophosphamide, bleomycin or tamoxifen.

Therapeutic drugs useful for treating CNS disorders can be administered in combination with each other. Combination chemotherapy treatment has advantages in that: (1) it avoids single agent resistance; (2) it can kill cells by different mechanisms; (3) by selecting drugs with nonoverlapping toxicities, each agent can be used at full dose. The precise dosage of a therapeutic agent to be delivered to a patient concomitantly with a antiglucocorticoid will be dependent upon the discretion and professional judgment of an attendant physician and will be in part dependent on such factors as the age, weight and particular neoplasia of the patient. The amount and precise regime will also depend on other factors including the severity of the condition to be treated. In general, if administered in combination with an antiglucocorticoid, the dosage necessary to achieve a therapeutic effect will be reduced. For example, carmustine, is usually administered intravenously at doses of 150-200 mg/m². When given in combination with other therapeutic agents, the dose is usually reduced by 25-50%. When given in combination with an antiglucocorticoid, the dose may be reduced even more.

The methods of the present invention may also be useful for treating CNS disorders such as CNS bacterial diseases. In some embodiments, bacterial diseases such as bacterial meningitis or bacterial abscess can be treated. A variety of different bacterial organisms cause bacterial meningitis including Streptococcus pneumonia, Streptococcus pyogenes, Staphylococcus aureu, Staphylococcus epidermidis, Enterobacteriacea, Propionibacterium, Pseudomonoas aeruginosa, Neisseria meningitis, Haemophilus influenzae and Listeria moncytogenes. One of skill in the art will know how to diagnose the different forms of bacterial meningitis and choose the appropriate antibacterial agent for administration with an antiglucocorticoid. The choice of antibacterial agent will depend on the identified or suspected causative organism. For example, in some embodiments of the present invention, penicillins, cephalosporins, monobactams, carbapenems, aminoglycosides, vancomycins, glycopeptides, tetracyclines, macrolides, sulfonamides and trimethoprim-trimethoprims or sulfamethoxazoles will be administered concomitantly with an antiglucocorticoid to treat CNS bacterial diseases. Examples include Penicillin G, ampicillin, nafcillin, ceftriaxone, ceftazidime, azactum, meropenem, gentamicin, vancomycin, chloramphenicol, doxycline, or erythromycin. The appropriate dose of antibacterial agents administered in combination with an antiglucocorticoid will again depend upon the nature and severity of the disease. For example, certain strains of bacteria are sensitive to differing amounts of antibacterial agent. Chloramphenicol, for example, may be effective against sensitive bacterial strains at 8 ug/ml or less whereas higher concentrations may be necessary for less sensitive bacterial strains. When administered in combination with an antiglucocorticoid, these concentrations may reduced. Combination drug therapy may be appropriate for treating CNS bacterial diseases. Chloramphenicol, for example, together with penicillin is useful for the treatment of brain abscesses. Chloramphenicol may also be substituted with other antibacterial agents including other aminoglycosides such as clindamycin or metronidazole. One of skill, using the methods of the present invention, will know how to increase the efficacy of the drug treatment and/or lower the amount of drug necessary for treatment by adding an antiglucocorticoid therapy.

In some embodiments, the methods of the present invention may also be useful for treating viral diseases. For example, patients having viral encephalitis including HSV encephalitis, cytomegalovirus encephalitis and varicella encephalitis may be treated with antiviral agents in combination with an antiglucocorticoid therapy. Using the methods of the present invention, one of skill in the art will know to administer an antiglucocorticoid in combination with an appropriate antiviral agents to treat patients having a viral disease. Antiviral agents may include acyclovir, ganciclovir and foscarnet. HIV encephalitis may also be treated by the methods of the present invention. Drugs such as acyclovir, ceftriaxone, pyrimethamine, sulfadiazine, clindamycin, flucytosine, doxycycline, ganciclovir and foscarnet can be used in combination with an antiglucocorticoid to effectively treat patients.

In some embodiments, the methods of the present invention may be useful for treating antifungal diseases. Examples of antifungal agents are amphotericin B, flucytosine, fluconazole, or itraconazole.

The methods of the present invention can also be used to treat CNS disorders such as neurodegenerative disorders or neuropsychiatric diseases. Disorders or diseases such as major depression, mania, bipolar manic-depressive illness, schizophrenia, schizoaffective disorder and panic and anxiety disorder can be treated using the methods of the present invention. Using these methods, the skilled practitioner will know how to administer an antiglucocorticoid with an antidepressant, anticonvulsant or antipsychotic. In some embodiments, therapeutic drugs useful for treating neuropsychiatric diseases include fluoxetine-selective serotonin reuptake inhibitors, amitriptyline-tricyclics, venlafaxine-bupropion, Haloperidol, Risperidone, Olanzapine, Lorazepam, Clonazepam, Buspirone, Valproic acid, Topiramate, Carbamazepine, or Lithium. Neurodegenerative disorders, such as Alzheimer's Disease, Parkinson's Disease, multiple sclerosis and seizure disorder can be treated by the methods of the present invention. Therapeutic drugs useful for treating Alzheimer's such as acetylcholinesterase inhibitors including donepezil, tacrine, rivastigmine and galantamine can be administered in combination with an antiglucocorticoid as well as any other drug that targets the pathophysiology of the disease. Therapeutic drugs, such as interferons and glatiramer acetate can be used with the methods of this invention for the treatment of multiple sclerosis. In some embodiments, the methods of the present invention can be used to treat Parkinson's disease by coadministering an antiglucocorticoid with a drug that targets the pathophysiology of Parkinson's. Other examples of drugs that can be used with this invention to treat Parkinson's include levodopa, carbidopa, benserazide, pergolide, bromocriptine, selegiline, amantadine, and trihexyphenidyl HCL. Methods of the present invention can also be used to treat seizure disorders or strokes. Drugs useful for coadministration with an antiglucocorticoid will include drugs aimed at decreasing the amount of tissue damage after an ischemic event or drugs including carbamazepine, fosphenytoin, valproic acid, phenytoin, felbamate, clonazepam, primidone, topiramate, ehthosuximide, gabapentin and phenobarbitol. The methods of the present invention can also be used to treat disorders such as epilepsy. Drugs useful for coadministration with an antiglucocorticoid will include antiepileptic and antiseizure drugs such as Carbamazepine and Valproate.

XI. Glucocorticoid Blocker Dosage Regimens

The methods of the invention increase BBB permeability and/or prevent or reduce glucocorticoid-induced decreases in BBB permeability. The amount of glucocorticoid blocker adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen may also take into consideration the pharmacokinetics, i.e., the glucocorticoid blockers' rate of absorption, bioavailability, metabolism, clearance, and the like (see, for example, Hidalgo-Aragones (1996) J. Steroid Biochem. Mol. Biol. 58:611-617; (Droning; (1996) Pharmazie 51:337-341; Fotherby (1996) Contraception 54:59-69; Johnson (1995) T Pharm. Sci. 84:1144-1146; Rohatagi (1995) Pharmazie 50:610-613; Brophy (1983) Eur. J. Clin. Pharmacol. 24:103-108; Remington's Pharmaceutical Science, 15th ed., Mack Publishing Company, Easton, Pa. (1980)).

The state of the art allows the clinician to determine the dosage regimen for each individual patient, glucocorticoid blocker, and disease or condition treated. Glucocorticoid blocker compounds suitable for use in the practice of this invention may be administered as single or multiple dosages. The example provided below for mifepristone can be used to guide the determination of the dosage regimen, including dosing schedule and dosage levels, of any glucocorticoid blocker administered when practicing the methods of the invention.

For example, a typical preferred pharmaceutical formulation for oral administration of mifepristone would be about 5 to 15 mg/kg of body weight per patient per day, more preferably between about 8 to about 12 mg/kg of body weight per patient per day, most preferably 10 mg/kg of body weight per patient per day, although dosages of between about 0.5 to about 25 mg/kg of body weight per day maybe used in the practice of the invention. Even wider range of dosages may be utilized in some instances, such as, for any of the other conventional methods for administering compounds to reach the CNS, e.g., admnistration into the blood stream. Actual methods for preparing parenterally administrable glucocorticoid blockers formulations will be known or apparent to those skilled in the art and are described in more detail in such publications as Remington's Pharmaceutical Science, 15th ed., Mack Publishing Company, Easton, Pa. (1980). At the preferred dosage of about 8 to 20 mg/kg of body weight per patient per day, administration can continue for a period of about 4 days. In an alternative dosing regimen, mifepristone may be administered in a daily amount of between about 300 mg/day to about 800 mg/day, more preferably about 600 mg/day.

When therapeutic agents such as anti-parasitic, anti-bacterial, anti-fungal, antitumor, anti-viral agents, anti-neurodegenerative, and anti-psychotic agents and the like are administered in combination with antiglucocorticoids, their ability to enter the CNS of patient and have therapeutic effect is increased. In some embodiments, when administered according to the methods of the present invention, the active therapeutic agent may be administered at a dosage that is at least about 10 fold less, often at least about 20 fold less and sometimes at least about 50 fold less than a control formulation in which the agent is administered by itself. An attendant physician treating a patient having a CNS disorder with the methods of the present invention will know how to adjust the precise dosage of a therapeutic drug administered concomitantly with an antiglucocorticoid in order to achieve a desired therapeutic effect. Generally, high dosages of therapeutic drugs are necessary for treatment of patients having CNS disorders because of the low permeability of the blood brain barrier. High dosages of therapeutic drugs, however, may be harmful because of toxic side effects. Typically, toxic side effects are dose dependent. When administered concomitantly with an antiglucocorticoid therapy, therapeutic drugs for treating patients with CNS disorders may be administered at smaller dosages thereby decreasing harmful side effects while increasing therapeutic effect. For example, the toxic effects of chemotherapeutic agents are well known, yet, chemotherapy is an essential treatment for cancer patients. Using the methods of the present invention, antiglucocorticoids can be administered concomitantly with chemotherapeutic agents thereby increasing the amount of chemotherapeutic drug entering the CNS of the patient while maintaining or reducing the amount of actual drug administered to a patient.

When an antiglucocorticoid is administered concomitantly with a therapeutic drug for use in the present invention, the therapeutic drug will reach a therapeutically effective level in the CNS when the blood-brain barrier is lowered by a blood-brain barrier lowering amount of an antiglucocorticoid. In one embodiment, to lower the permeability of the blood brain barrier to the appropriate permeability level, the antiglucocorticoid will be administered once up to two weeks prior to administration of the therapeutic drug. In another embodiment, the antiglucocorticoid will be administered more than once for a two week time period prior to administration of the therapeutic drug. In some embodiments, the antiglucocorticoid may be administered at the same time as the therapeutic drug or several hours before or after administration of the therapeutic drug. The antiglucocorticoid may also be delivered as microspheres administered transdermally or intradermally for slow release in the body. An attendant physician will know how to determine when a therapeutic drug has reached a therapeutically effective level in the CNS blood brain barrier permeability by known methods, e.g., clinical exam of a patient, or by measuring drug levels in the cerebrospinal fluid.

XII. Glucocorticoid Blocker Kits

After a pharmaceutical comprising a glucocorticoid blocker has been formulated in a suitable carrier, it can be placed in an appropriate container and labeled for treatment of an indicated disease. Additionally, another pharmaceutical comprising at least one other therapeutic agent useful in the treatment of a disease of the CNS will be placed in the container as well, and labeled for treatment of the indicated disease. Alternatively, a single pharmaceutical comprising a glucocorticoid blocker and at least one other therapeutic agent useful in the treatment of a disease of the CNS can be placed in an appropriate container and labeled for treatment of an indicated disease. For administration of pharmaceuticals comprising glucocorticoid blockers and of pharmaceuticals comprising, in a single pharmaceutical, glucocorticoid blockers and at least one other therapeutic agent useful in the treatment of a disease of the CNS, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

In one embodiment, the invention provides for a kit for the treatment of a disease of the CNS, which includes a glucocorticoid blocker and instructional materials teaching the indications, dosage, and schedule of administration of the glucocorticoid blocker. When mifepristone is the glucocorticoid blocker provided in the kit, the instructional material indicates that the glucocorticoid blocker can be used in a daily amount of about 8 to 12 mg/kg of body weight per day, and the administration of the glucocorticoid blocker continues for a period of about four days.

In the light of the foregoing, and of the examples presented below, it will be understood by one of ordinary skill in the art that administration of a glucocorticoid blocker may be for a longer or a shorter period of time than four days, and, if concomitantly administered with another drug, that the glucocorticoid blocker may be given at the same time, or may be administered beginning minutes, hours, or days before or after administration of the other drug depending on the characteristics of the particular compounds and the status of the patient.

### EXAMPLES

### Example 1

### Corticosteroid Administration Decreases BBB Permeability

Adrenalectomized rats (male Sprague Dawley 175-200 grams) were implanted with drug-release pellets (Innovative Research of America, Sarasota, Fla.) and maintained, 3 rats per cage, under controlled temperature and lighting (12 hours light/12 hours dark) with food and water ad libitum. The implanted pellets contained either 100 mg corticosterone (for haloperidol experiments), 50 mg corticosterone (for clozapine experiments) or placebo. Two hours prior to sacrifice the animals were injected with either haloperidol (1 mg/kg s.c; RBI Natick, Mass.) or an equivalent volume of vehicle (0.3% tartaric acid, pH 5.3) or with either clozapine (15 mg/kg s.c.; RBI, Natick, Mass.) or vehicle (0.9% saline plus 0.8% acetic acid).

Animals were sacrificed by decapitation during the first four hours of the light cycle, blood collected and brains removed and frozen on dry ice and stored at −80° C. Corticosterone was measured in plasma by radioimmunoassay (ICN Biochem, Costa Mesa, Calif.) to confirm adrenalectomy and corticosterone replacement. Frozen brains were sliced into 250 μm sections with a cryostat. The medial prefrontal cortex (AP 13.7 to 12.2 mm) was dissected with a scalpel, the striatum (AP 10.7 to 9.7 mm) removed with stainless steel cannulae from frozen slices and the core and shell of the nucleus accumbens (AP 10.7 to 9.7 mm) was removed. Brain regions were dissected within 24-72 hours of slicing. Tissue was placed in 0.1 M perchloric acid with 0.1 mM EDTA and stored for no longer than 2 weeks at −80° C.

Dopamine metabolites homovanillic acid (HVA) and dihydroxyphenylacetic acid (DOPAC) were measured in the brains of the experimental animals. Samples were thawed, homogenized by sonication in 0.1 M perchloric acid/0.1 mM EDTA, and centrifuged for 2 minutes. Tissue pellets were dissolved in 1.0 N NaOH for protein determination (BioRad, Richmond, Calif.). Cortical supernatants were filtered through a 0.45 μm filter and 5-80 μl of supernatant was injected directly onto a C18 reverse phase analytical column (5 μm, 250×4.6 mm; Biophase ODS, BAS, West Lafayette, Ind.) protected by a precolumn cartridge (5 μm, 30×4.6 mm, BAS) as described with modification (Lindley et al. Proc. Soc. Exp. Biol. Med. 188:282-286 (1988)). DOPAC and HVA were detected using an electrochemical detector. For cortical regions, the conditioning electrode was set at +0.35 V and the dual analytical electrode was set at +0.02 V and −0.35 V, respectively (ESA, Bedford, Mass.). For other regions a single analytical electrode set at +0.72 V was used (BAS, West Lafayette, Ind.). Brain clozapine levels were analyzed by National Medical Services, Inc. (Willow Grove, PN) while brain and plasma haloperidol and reduced haloperidol and plasma clozapine levels were analyzed by Analytical Psychopharmacology Laboratories (Nathan Kline Institute, Orangeburg, N.Y.), both by gas chromatography.

Consistent with prior work demonstrating that both haloperidol and clozapine increase dopamine utilization in the brain, measured levels of HVA and DOPAC were elevated in the brains of vehicle-treated animals. However, the effects of haloperidol and clozapine on dopamine metabolite levels were smaller in corticosterone-treated animals than in animals receiving vehicle pellets. In addition, corticosterone-treatment also significantly decreased brain concentrations of haloperidol, the reduced form of haloperidol, and clozapine without decreasing plasma reduced haloperidol or plasma clozapine levels. Thus, corticosterone inhibits both haloperidol-induced and clozapine-induced increases in dopamine metabolite levels in the brain.

### Example 2

### Glucocorticoid Blocker-Induced Increase in Permeability of the BBB

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10-day sustained-release pellet. Two hours prior to sacrifice the animals are injected with either haloperidol (1 mg/kg s.c; RBI Natick, Mass.) or an equivalent volume of vehicle (0.3% tartaric acid, pH 5.3) or with either clozapine (15 mg/kg s.c.; RBI, Natick, Mass.) or vehicle (0.9% saline plus 0.8% acetic acid).

Animal sacrifice is by decapitation during the first four hours of the light cycle. Blood is collected and brains removed and frozen on dry ice for storage at −80° C. Frozen brains are sliced into 250 μm sections with a cryostat. The medial prefrontal cortex (AP 13.7 to 12.2 mm) is dissected with a scalpel, the striatum (AP 10.7 to 9.7 mm) is removed with stainless steel cannulae from frozen slices and the core and shell of the nucleus accumbens (AP 10.7 to 9.7 mm) is removed. Brain regions are dissected within 24-72 hours of slicing. Tissue is placed in 0.1 M perchloric acid with 0.1 mM EDTA for storage for no longer than 2 weeks at −80° C.

Dopamine metabolites homovanillic acid (HVA) and dihydroxyphenylacetic acid (DOPAC) are measured in the brains of the experimental animals. Samples are thawed and are homogenized by sonication in 0.1 M perchloric acid/0.1 mM EDTA, and centrifuged for 2 minutes. Tissue pellets are dissolved in 1.0 N NaOH for protein determination (BioRad, Richmond, Calif.). Cortical supernatants are filtered through a 0.45 μm filter and 5-80 μl of supernatant is injected directly onto a C18 reverse phase analytical column (5 μm, 250×4.6 mm; Biophase ODS, BAS, West Lafayette, Ind.) protected by a precolumn cartridge (5 μm, 30×4.6 mm, BAS) as described with modification (Lindley et al. Proc. Soc. Exp. Biol. Med. 188:282-286 (1988)). DOPAC and HVA were detected using an electrochemical detector. For cortical regions, the conditioning electrode is set at +0.35 V and the dual analytical electrode is set at +0.02 V and −0.35 V, respectively (ESA, Bedford, Mass.). For other regions a single analytical electrode set at +0.72 V is used (BAS, West Lafayette, Ind.). Brain clozapine levels are analyzed by National Medical Services, Inc. (Willow Grove, PN) while brain and plasma haloperidol and reduced haloperidol and plasma clozapine levels are analyzed by Analytical Psychopharmacology Laboratories (Nathan Kline Institute, Orangeburg, N.Y.), both by gas chromatography.

Levels of haloperidol, clozapine and the dopamine metabolites homovanillic acid (HVA) and dihydroxyphenylacetic acid (DOPAC) are measured in the brains of the experimental animals. Measured levels of HVA and DOPAC are elevated in the brains of vehicle-treated animals. The increase in dopamine metabolite levels following haloperidol and clozapine treatment, as well as the brain concentrations of haloperidol and clozapine, are greater in mifepristone-treated animals than in animals receiving placebo. This demonstrates glucocorticoid blocker-induced increases in haloperidol and clozapine levels in the brain and potentiation of haloperidol and clozapine-induced increases in dopamine metabolite levels in the brain, consistent with an increase in BBB permeability due to mifepristone.

### Example 3

### Glucocorticoid Blocker-Induced Increase in Permeability of the BBB and Resulting Increase in Delivery of Amphotericin B

Amphotericin B is a polyene antibiotic with potent antifungal activity.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10-day sustained release pellet. Two hours prior to sacrifice the animals are injected with either Amphotericin B (0.1 mg i.v.; Sigma Chemical Co., (800) 325-3010) or an equivalent volume of vehicle (0.1% DMSO in saline, pH 11).

Animal sacrifice is by decapitation during the first four hours of the light cycle. Blood is collected and brains removed and frozen on dry ice for storage at −80° C. Amphotericin B concentration is measured in the brains of the experimental animals. The Amphotericin B concentration is greater in the brains of mifepristone-treated animals than in placebo-treated animals. This result demonstrates a glucocorticoid blocker-induced increase in Amphotericin B delivery to the brain, consistent with an increase in BBB permeability due to mifepristone.

### Example 4

### Glucocorticoid Blocker-Induced Increase in Permeability of the BBB and Resulting Increase in Delivery of Ampicillin

Ampicillin (D[-]-α-Aminobenzylpenicillin) is a potent antibacterial agent structurally related to penicillin.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10-day sustained release pellet. Two hours prior to sacrifice the animals are injected with either ampicillin (1.5 mg i.v.; Sigma Chemical Co., (800)325-3010) or an equivalent volume of vehicle (0.9% sodium chloride colution).

Animal sacrifice is by decapitation during the first four hours of the light cycle. Blood is collected and brains removed and frozen on dry ice for storage at −80° C. Ampicillin concentration is measured in the brains of the experimental animals. The ampicillin concentration is greater in the brains of mifepristone-treated animals than in placebo-treated animals. This result demonstrates a glucocorticoid blocker-induced increase in ampicillin delivery to the brain, consistent with an increase in BBB permeability due to mifepristone.

### Example 5

### Glucocorticoid Blocker-Induced Increase in Permeability of the BBB and Resulting Increase in Delivery of Methotrexate

Methotrexate (N-[4-[[)2,4-Diamino-6-pteridinyl-)methylamino]benzoyl]-L-glutamic acid) is a folic acid antagonist that is a potent cancer chemotherapy agent.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10 day sustained-release pellet. Two hours prior to sacrifice the animals are injected with either methotrexate (0.5 mg i.v; Sigma Chemical Co., (800)325-3010) or an equivalent volume of vehicle (saline, pH 9).

Animal sacrifice is by decapitation during the first four hours of the light cycle. Blood is collected and brains removed and frozen on dry ice for storage at −80° C. Methotrexate concentration is measured in the brains of the experimental animals. The methotrexate concentration is greater in the brains of mifepristone-treated animals than in placebo-treated animals. This result demonstrates a glucocorticoid blocker-induced increase in methotrexate delivery to the brain, consistent with an increase in BBB permeability due to mifepristone.

### Example 6

### Glucocorticoid Blocker-Induced Increase in Permeability of the BBB and Resulting Increase in Delivery of Adriamycin

Adriamycin ((8S-cis)-10-(3-Amino-2,3,6-Trideoxy-alpha-L-Lyxo-Hexopyranosyl)Oxy-7,8,9,10-Tetrahydro-6,8,11-Trihydroxy-8-(Hydroxyacetyl)-1-Methoxy-5,12-Naphthacenedione, also known as doxorubicin) is a potent cancer chemotherapy agent.

Rats (male Sprague Dawley 175-200 grams) are maintained, 3 rats per cage, under controlled temperature and lighting (12 hours light/12 hours dark) with food and water ad libitum. Four hours prior to sacrifice rats are injected with mifepristone (dissolved in benzyl benzoate-sesame oil(1:4) with slight warming; dosage 2 mg s.c.) or placebo. Two hours prior to sacrifice the animals are injected with either adriamycin (0.5 mg i.v; “doxorubicin hydrochloride,” Sigma Chemical Co., (800)325-3010) or an equivalent volume of vehicle (saline, pH 9).

Animal sacrifice is by decapitation during the first four hours of the light cycle. Blood is collected and brains removed and frozen on dry ice for storage at −80° C. Adriamycin concentration is measured in the brains of the experimental animals. The adriamycin concentration is greater in the brains of mifepristone-treated animals than in placebo-treated animals. This result demonstrates a glucocorticoid blocker-induced increase in adriamycin delivery to the brain, consistent with an increase in BBB permeability due to mifepristone.

All publications and patent applications cites in this specification are herein incorporated by reference as if each individual publication or patent application were specifically and individually indicated to be incorporated by reference.

Although the foregoing invention has been described in some detail by way of illustration and examples for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of increasing the permeability of the blood brain barrier in a patient having a CNS disorder amenable to drug therapy and not otherwise indicative of an antiglucocorticoid therapy wherein the method comprises:
administering an antiglucocorticoid drug and administering a therapeutic drug to the patient having a CNS disorder amenable to drug therapy and not otherwise indicative of an antiglucocorticoid therapy, wherein the amount of antiglucocorticoid administered is sufficient to increase the permeability of the blood brain barrier to the therapeutic drug.

2. A method of increasing the amount of a therapeutic drug delivered to the CNS of a patient having a CNS disorder amenable to drug therapy and not otherwise indicative of an antiglucocorticoid therapy wherein the method comprises:
administering an antiglucocorticoid drug and administering a therapeutic drug to the patient having a CNS disorder amenable to drug therapy and not otherwise indicative of an antiglucocorticoid therapy, wherein the amount of antiglucocorticoid administered is sufficient to increase the permeability of the blood brain barrier to the therapeutic drug.

3. The method of claim 2, wherein the CNS disorder is a neoplastic disease, bacterial disease, viral disease, fungal disease, neuropsychiatric disease or neurodegenerative disorder.

4. The method of claim 3, wherein the CNS disorder is a neoplastic disease and the therapeutic drug is a chemotherapeutic agent.

5. The method of claim 4, wherein the chemotherapeutic agent is administered in combination with radiation therapy.

6. The method of claim 4, wherein the neoplastic disease is a cerebral metastases or malignant astrocytoma.

7. The method of claim 6, wherein the chemotherapeutic agent is a nitrosoureas.

8. The method of claim 7, wherein the nitrosoureas is selected from the group consisting of lomustine, semustine and carmustine.

9. The method of claim 4, wherein the CNS disorder is a cerebral metastases and the chemotherapeutic agent is selected from the group consisting of microtubilin inhibitors, topoisomerase inhibitors, antimicrobial agents and platinum compounds.

10. The method of claim 9, wherein the chemotherapeutic agent is vinblastine, etoposide, topotecan, penicillin, or cisplatin.

11. The method of claim 4, wherein the CNS disorder is a cerebral metastases and the chemotherapeutic agent is selected from the group consisting of antimetabolites, DNA damaging agents, endocrine agents and anti-tumor antibiotics.

12. The method of claim 11, wherein the chemotherapeutic agent is methotrexate, cyclophosphamide, bleomycin or tamoxifen.

13. The method of claim 3, wherein the CNS disorder is a bacterial disease and the bacterial disease is bacterial meningitis or a CNS bacterial abscess.

14. The method of claim 13, wherein the therapeutic drug is selected from the group consisting of penicillins, cephalosporins, monobactams, carbapenems, aminoglycosides, glycopeptides, tetracyclines, macrolides, sulfonamides, trimethoprims, and chloramphenicol.

15. The method of claim 14, wherein the bacterial disease is bacterial meningitis and the therapeutic drug is selected from the group consisting of chloramphenicol, ampicillin, cefotaxime, ceftriaxone and fectizoxime.

16. The method of claim 14, wherein the bacterial disease is a CNS bacterial abscess and the therapeutic drug is selected from the group consisting of chloramphenicol, penicillin, and metronidazole.

17. The method of claim 3, wherein the CNS disorder is viral encephalitis.

18. The method of claim 17, wherein viral encephalitis includes HSV encephalitis, cytomegalovirus encephalitis, and varicella encephalitis.

19. The method of claim 18, wherein the therapeutic drug is selected from the group consisting of acyclovir, ganciclovir, and foscarnet.

20. The method of claim 3, wherein the CNS disorder is HIV encephalitis and the therapeutic drug is selected from the group consisting of acyclovir, ceftriaxone, pyrimethamine, sulfadiazine, clindamycin, flucytosine, doxycycline, ganciclovir, and foscarnet.

21. The method of claim 3, wherein the CNS disorder is a neuropsychiatric disease selected from the group consisting of psychotic disorder and affective disorder.

22. The method of claim 21, wherein the neuropsychiatric disease is an affective disorder selected from the group consisting of major depression, mania, and bipolar manic-depressive illness and the therapeutic drug is an antidepressant, anti-convulsant or antipsychotic agent.

23. The method of claim 22, wherein the therapeutic drug is selected from the group consisting of luoxetine-selective serotonin reuptake inhibitors, amitriptyline-tricyclics, venlafaxine-bupropion, Haloperidol, Risperidone, Olanzapine, Lorazepam, Clonazepam, Buspirone, Valproic acid, Topiramate, Carbamazepine, and Lithium.

24. The method of claim 21, wherein the neuropsychiatric disease is a psychotic disorder selected from the group consisting of schizophrenia, schizoaffective disorder and panic/anxiety disorder and the therapeutic drug is an antidepressant, anticonvulsant or antipsychotic agent.

25. The method of claim 24, wherein the therapeutic drug is selected from the group consisting of fluoxetine-selective serotonin reuptake inhibitors, amitriptyline-tricyclics, venlafaxine-bupropion, Haloperidol, Risperidone, Olanzapine, Lorazepam, Clonazepam, Buspirone, Valproic acid, Topiramate, Carbamazepine, and Lithium.

26. The method of claim 3, wherein the CNS disorder is a neurodegenerative disorder and the neurodegenerative disorder is selected from the group consisting of Alzheimer's Disease, multiple sclerosis, Parkinson's Disease, and seizure disorder.

27. The method of claim 26, wherein the neurodegenerative disorder is Alzheimer's Disease and the therapeutic drug is an acetylcholinesterase inhibitor.

28. The method of claim 26, wherein the neurodegenerative disorder is multiple sclerosis and the therapeutic drug is selected from the group consisting of Interferon-1b, Interferon-1a, and glatiramer acetate.

29. The method of claim claim 26, wherein the neurodegenerative disorder is seizure disorder and the therapeutic drug is selected from the group consisting of carbamazepine, fosphenytoin, valproic acid, phenytoin, felbamate, clonazepam, primidone, topiramate, ehthosuximide, gabapentin and phenobarbitol.

30. The method of claim 26, wherein the neurodegenerative disorder is Parkinson's Disease and the therapeutic drug is selected from the group consisting of levodopa, carbidopa, benserazide, pergolide, bromocriptine, selegiline, amantadine, and trihexyphenidyl HCL.

31. The method of claim 3, wherein the CNS disorder is fungal.

32. The method of claim 31, wherein the CNS disorder is fungal and the therapeutic drug is selected from the group consisting of amphotericin B, flucytosine, fluconazole, and itraconazole.

33. A kit for the treatment of a patient having a CNS disorder amenable to drug therapy and not otherwise indicative of an antiglucocorticoid therapy, the kit comprising an antiglucocorticoid in sufficient amount to increase permeability of the patient's blood brain barrier, a therapeutically effective amount of a drug useful for treating the CNS disorder, and instructions for the concomitant administration of the drug and the antiglucocorticoid.

34. The kit of claim 33, wherein the CNS disorder is a neoplastic disease, bacterial disease, viral disease, fungal disease, neuropsychiatric disease, or neurodegenerative disorder.
